(19) United States
(12) Patent Application Publication
(10) Pub. No.: US 2014/0066862 A1
(43) Pub. Date: Mar. 6, 2014

(54) ERGONOMIC SYRINGE SYSTEMS

(71) Applicants: Thomas Lloyd BELLAIRE, Burnaby (CA); Dennis SCHWEERS, Burnaby (CA); Richard HALL, Port Moody (CA); Brian KEANE, Vancouver (CA)

(72) Inventors: Dennis Schweers, Burnaby (CA); Richard Hall, Vancouver (CA); Brian Keane, Vancouver (CA); Thomas Lloyd Bellaire, Burnaby (CA)

(73) Assignee: BRITISH COLUMBIA CANCER AGENCY BRANCH, Vancouver, BC (CA)

(21) Appl. No.: 14/115,857

(22) PCT Filed: May 18, 2012
(86) PCT No.: PCT/CA2012/050333

§ 371 Date: Nov. 5, 2013

Related U.S. Application Data
(60) Provisional application No. 61/488,258, filed on May 20, 2011.
(60) Provisional application No. 61/536,643, filed on Sep. 20, 2011.

(51) Int. Cl. A61M 5/31; A47C 1/00; A61M 5/00

(52) U.S. Cl. CPC A61M 5/3137; A61M 5/008; A47C 1/00
USPC 604/227; 248/205.1; 297/188.01; 294/137

(57) ABSTRACT

Ergonomic syringe systems provide attachments that may be used to allow users to dispense or draw in fluid with increased operator comfort and reduced risk of repetitive strain injuries. The disclosure describes supports for holding syringes that permit one-handed operation, handles that are removably attachable to syringe plungers and hand supports that are removably attachable to syringe barrels.

### REFERENCE TO RELATED APPLICATIONS

This application claims priority from U.S. application No. 61/488,258, filed 20 May 2011, and entitled ERGONOMIC SYRINGE ADAPTER and U.S. application No. 61/536,643 filed 20 Sep. 2011 and entitled ERGONOMIC SYRINGE ADAPTER both of which are hereby incorporated herein by reference for all purposes. For purposes of the United States of America, this application claims the benefit under 35 U.S.C. §119 of U.S. application No. 61/488,258, filed 20 May 2011, and entitled ERGONOMIC SYRINGE ADAPTER and U.S. application No. 61/536,643 filed 20 Sep. 2011 and entitled ERGONOMIC SYRINGE ADAPTER both of which are hereby incorporated herein by reference for all purposes.

### TECHNICAL FIELD

This invention relates to syringes. Specific embodiments provide apparatus for facilitating operation of syringes. An example embodiment provides a chemotherapy treatment chair having an arm rest and a syringe holder positioned to support a syringe while a medical professional operates the syringe to inject chemotherapy drugs into a patient. Other embodiments provide grips/adapters that may be mounted to the barrel and/or plunger of a syringe to provide a more ergonomic way to use the syringe.

### BACKGROUND

FIG. 1 shows a typical prior art syringe 10. Syringe 10 comprises a body or barrel 12 and a plunger 14. A sealing piston 15 is engaged in a bore of barrel 12 so that advancing the plunger 14 toward a tip 16 of the syringe causes ejection of fluids from the syringe through an opening 17 on the distal end of the syringe. Syringe 10 may comprise a fitting, for example, a Luer lock fitting or any other suitable fitting for coupling syringe 10 to a needle, tube or other conduit for carrying the fluid expelled from syringe 10.

Syringes like syringe 10 are designed to be held in one hand by a user who presses their thumb on a pad 19 on the end of plunger 14 and who holds tabs 20 which project on either side of barrel 12 with the first and second fingers of the hand holding the syringe. The user can then advance the plunger by pressing with their thumb. The user can monitor the progress of the injection (e.g. determine how much fluid has been injected) with reference to graduations or other indicia 18 on barrel 12. Barrel 12 is typically made of a translucent or transparent plastic, glass, or other suitable material such that the position of piston 15 can be viewed through all or a portion of barrel 12. FIG. 1A shows a syringe being used in a conventional manner to inject fluids into a patient.

Some patients are treated for cancer using periodic injections of chemotherapy drugs. The chemotherapy drugs are typically injected by a medical professional who monitors to ensure that the injection is going well. In many cases chemotherapy requires injections of relatively large volumes of drugs. Some such chemotherapy drugs are toxic and/or corrosive. It can be important not to inject the drugs too quickly into the patient. As a result of the above, medical personnel who inject chemotherapy drugs may spend a significant portion of each day holding relatively heavy syringes while maintaining constant thumb pressure to inject chemotherapy drugs at an appropriate rate into the patients they are helping to treat. This can result in repetitive strain injuries or other injuries to the health care providers.

The inventors have conducted an ergonomic assessment of the task performed by chemotherapy nurses. This assessment revealed that administration of chemotherapy drugs via syringe (20 ml or greater size) results in a significant risk for injury to the hands/fingers, wrist, forearm and elbow of the chemotherapy nurses. This is likely due to prolonged hand grip force combined with awkward hand/finger postures. Extended use of syringes can result in significant injury to operators of syringes in other fields as well.

Another group of professionals who make extensive use of syringes are pharmacy personnel who may be charged with loading multiple syringes with drugs. Pharmacy personnel have reported injuries of the wrist, hand, elbow (medial and lateral sides), and shoulder. These injuries may result in symptoms such as pain, tenderness, stiffness, tingling, and muscle spasms. Many pharmacy personnel experience pain or discomfort in one or more of the following areas: hand, wrist, forearm, elbow, shoulder. This pain or discomfort is often related to the actions of withdrawing solutions (pulling plunger of syringe) and injecting solutions (depressing plunger of syringe).

Risk factors for injuries related to the repetitive use of syringes include: repetitive and forceful gripping combined with awkward hand and wrist postures when pre-filling syringes; awkward and static shoulder posture when holding vials during the syringe pre-filling procedures; and awkward and repetitive hand and wrist postures combined with forceful gripping when injecting solutions into saline-filled IV bags.

Patents and applications relating to syringes and attachments for syringes include the following:
US 2012/0041388;
US 2011/0137260;
US 2011/0092952;
US 2009/0093787;
US 2007/0052409;
US 2004/0039345;
U.S. Pat. No. 7,118,556;
U.S. Pat. No. 6,616,634;
U.S. Pat. No. 6,368,308;
U.S. Pat. No. 6,312,410;
U.S. Pat. No. 5,584,814; and,
U.S. Pat. No. 5,554,132.

There remains a need for methods and apparatus which can alleviate the risk of injury from prolonged use of syringes. There is a particular need for such apparatus and methods that can be implemented at relatively low cost.

### SUMMARY

This invention has a range of aspects that may be used together in various combinations or independently. One aspect provides supports for supporting syringes in use. The supports may facilitate one-handed operation of supported syringes and may be adjustable for operator comfort and safety. Another aspect provides attachment handles that may be affixed to a syringe plunger to facilitate safe and comfortable operation of the syringe. Another aspect provides grips that are affixable to the barrels of syringes to facilitate safe and comfortable operation of the syringe.

One aspect provides an apparatus for supporting a syringe. The apparatus comprises a base configured for mounting to a support, a coupling configured for removable attachment to a proximal end of a plunger of the syringe; and, an arm connecting the coupling to the base. A user can operate the syringe with one hand by moving a barrel of the syringe relative to the base. The base may be a attachable to a table, tray, arm, or the like or may be incorporated into a piece of furniture. In an example embodiment the apparatus comprises a patient chair comprising a seat for a patient to sit in and an arm rest mounted adjacent to the seat wherein the base is attached to the arm rest.

In some embodiments the coupler comprises a T-slot arranged to receive a pad at the proximal end of the plunger. The arms of the T-slot may have a dimension about equal to a thickness of the pad such that the pad is a reasonably snug fit into the T-slot.

The coupler may comprise a retainer member having a first position wherein the retainer member projects to block removal of the pad from the T-slot and a second position wherein the retainer member does not block removal of the pad from the T-slot. A transverse dimension of the T-slot and a longitudinal dimension between the retainer member and a closed end of the T-slot may be substantially equal to facilitate good support of a syringe wherein the pad is circular or generally circular (for example, square with rounded corners).

The apparatus optionally but preferably comprises a hand support shaped to provide a passage for receiving a barrel of the syringe. The hand support may be resiliently compressible. In an example embodiment, the hand support has a smaller-diameter end and a larger-diameter end and the hand support tapers smoothly toward the smaller-diameter end. The hand support may be mounted to the barrel of a syringe such that the smaller-diameter end faces the tip (distal end) of the syringe barrel. The hand support may be generally circular in cross-section at the smaller-diameter end and flattened at the larger-diameter end.

In some embodiments the hand support has a major diameter and a minor diameter at the larger-diameter end and a ratio of the major diameter to the minor diameter is approximately 4:3. The major diameter may be, for example, approximately 2 inches (5 cm). In some embodiments, both the major diameter and the minor diameter are at least 1¼ inches (about 3.2 cm).

Another aspect provides hand supports for use with syringes. The hand supports comprise a body shaped to provide a through-passage for receiving a barrel of the syringe. the body has a smaller-diameter end and a larger-diameter end and the hand support tapers smoothly toward the smaller-diameter end. The body may be resiliently compressible. For example, the body may be made of a foam, memory foam, compressible rubber, or the like. In some embodiments the body is generally circular in cross-section at the smaller-diameter end and is flattened at the larger-diameter end. In some embodiments the body has a major diameter and a minor diameter at the larger-diameter end and a ratio of the major diameter to the minor diameter is approximately 4:3. The major diameter may be, for example, approximately 2 inches (5 cm). In some embodiments both the major diameter and the minor diameter are at least 1¼ inches (about 3.2 cm).

Another aspect provides an accessory handle for a syringe. The accessory handle comprises a handle connected to a coupling. The coupling is configured to engage a pad at a proximal end of a syringe plunger. The coupling comprises a T-slot and a retainer member having a first position wherein the retainer member projects to block removal of the pad from the T-slot and a second position wherein the retainer member does not block removal of the pad from the T-slot. The handle may have any of a range of designs as described herein.

Other aspects provide the use of apparatus as described herein in injecting chemotherapy or other drugs and methods for injecting and/or transferring fluids using syringe accessories as described herein.

Further aspects and example embodiments are illustrated in the accompanying drawings and/or described in the following description.

### BRIEF DESCRIPTION OF THE DRAWINGS

The accompanying drawings illustrate non-limiting example embodiments of the invention.

FIG. 1 shows a conventional syringe.

FIG. 1A shows a syringe being used to inject fluids into a patient.

FIG. 2 is a side view of a syringe support according to an example embodiment.

FIG. 3 is a side view of a syringe support according to another example embodiment.

FIG. 3A is a perspective view of a hand support.

FIG. 3B is an end view of the hand support of FIG. 3A.

FIG. 3C illustrates a support like that shown in FIG. 3 being used to inject fluids into a patient.

FIG. 4 is an enlarged perspective view of a coupling for connecting a syringe plunger to a syringe support.

FIG. 4A is a cross sectional view of the coupling of FIG. 4.

FIG. 5 is a partially cut-away side view of a syringe mounted to apparatus comprising an extension arm.

FIG. 5A is a side view of the syringe and support apparatus of FIG. 5 in use.

FIGS. 6A and 6B re respectively views showing syringes equipped with a cylindrical handle and a flat handle.

FIG. 7 is a perspective view of a bulb-like hand adapter that may be affixed removably to a proximal end of a syringe plunger.

FIGS. 7A and 7B are cross sectional views of the hand adapter of FIG. 7.

FIG. 7C illustrates a handle like that shown in FIG. 7 in use for filling a syringe.

FIG. 8 is a view showing an alternative hand adapter.

### DESCRIPTION

Throughout the following description specific details are set forth in order to provide a more thorough understanding to persons skilled in the art. However, well known elements may not have been shown or described in detail to avoid unnecessarily obscuring the disclosure. The following description of examples of the technology is not intended to be exhaustive or to limit the system to the precise forms of any example embodiment. Accordingly, the description and drawings are to be regarded in an illustrative, rather than a restrictive, sense.

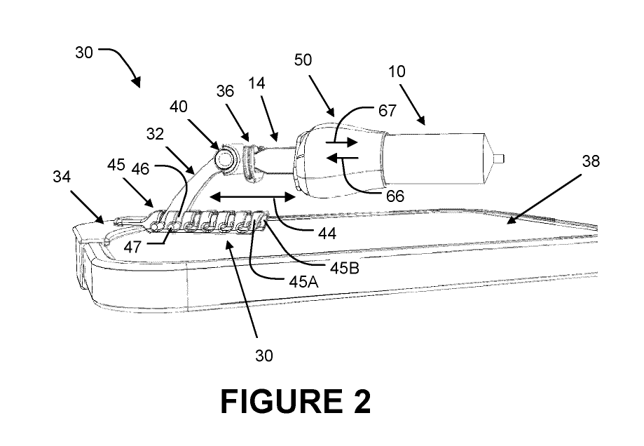

FIG. 2 is a side elevation view of a syringe 10 being held in a ergonomic syringe support 30 according to an example embodiment of the invention. Support 30 includes a arm 32 coupled between a base 34 and a coupling 36. Coupling 36 is adapted to receive and hold the proximal end of plunger 14 of syringe 10.

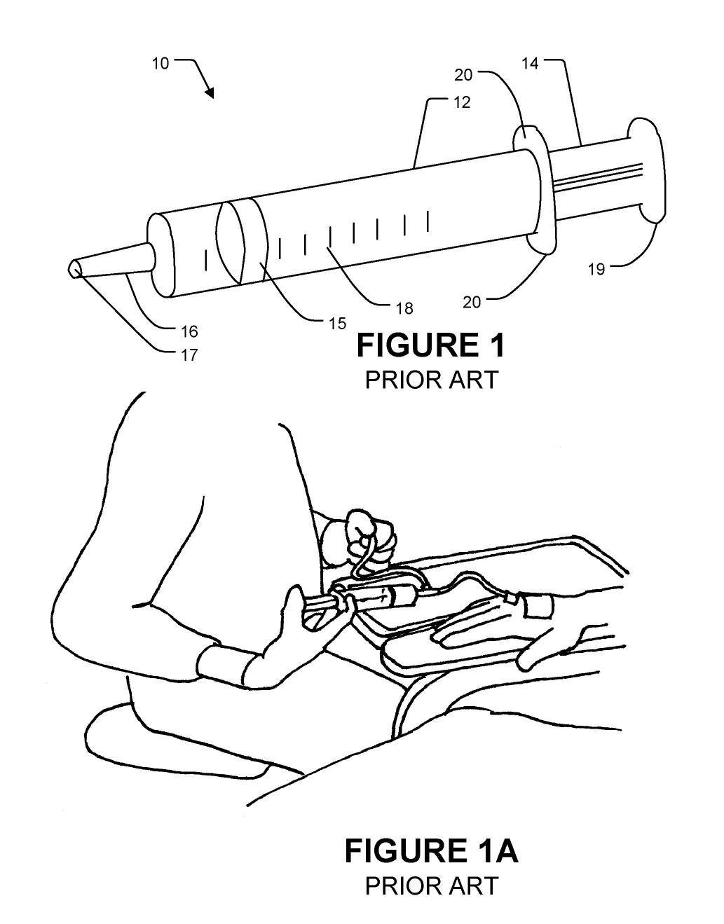

As shown in FIG. 1, many currently-available syringes 10 have a circular pad 19 at the proximal end of plunger 14. Coupling 36 may be configured, as described in more detail below, to receive and hold the circular pad 19 at the end of plunger 14 of syringe 10. In alternative embodiments, coupling 36 may be adapted to engage syringes having a non-standard end for engagement by coupling 36. For example, syringes for use with apparatus as described herein may be supplied with plungers having proximal ends specifically configured for releasable coupling to a coupling 36. In such embodiments, the proximal end of the syringe plunger and coupling 36 may be mating parts of any suitable coupling type. Such embodiments may have the disadvantage of not being usable with standard syringes and the advantage that the configuration of the syringe plunge may be selected to discourage use of the syringe except together with support 30 (or a suitable handle coupled to the plunger by way of a coupling 36 as described below).

As shown in FIG. 2, support 30 is adapted to support syringe 10 at a convenient location and orientation above a surface on which base 34 is sitting. For example, the embodiment illustrated in FIG. 2 shows a case where base 34 is mounted to an arm support tray 38 of a chemotherapy treatment chair (the rest of the chemotherapy treatment chair is not shown).

In the illustrated embodiment, coupler 36 is connected to arm 32 by way of a pivotal connection 40. Pivotal connection 40 may be locked at any desired angle to hold syringe 10 at the desired angle. Preferably pivotal connection 40 can at least support syringe 10 in a generally horizontal orientation as shown in FIG. 2 (e.g. a longitudinal centerline of syringe 10 is parallel to arm support 38) and an orientation in which syringe 10 is inclined upwardly (e.g. the distal end of syringe 10 is above the proximal end of syringe 10). In an example embodiment, pivotal connection 40 permits the angle of syringe 10 relative to surface 38 to be adjusted in a range of at least 0 degrees (parallel to surface 38) to 45 degrees (inclined upward so that the distal end of syringe 10 projects away from surface 38 at an angle of 45 degrees).

Pivotal connection 40 may be adjusted for the comfort of the person administering drugs using syringe 10.

Some embodiments provide a plurality of couplings dimensioned to couple to a plurality of different sizes of syringe plunger. A user may attach a selected one of the couplings to the arm to support a syringe. In some embodiments the pivotal connection 40 comprises two separable parts. One of the separable parts is attached to the arm. Each of the plurality of couplings is attached to a second part of the pivotal connection that can be snapped into place on or otherwise mated to the first part of the arm such that any selected one of the plurality of couplings may be pivotally coupled to the arm at the pivotal connection.

In the embodiment illustrated in FIG. 2, support 30 permits adjustment of the location of syringe 10 in a direction 44. Syringe 10 may be locked in place at a desired location in direction 44. In the illustrated embodiment, this adjustability is provided by a clip-track 45 made up of two members 45A each provided with notches 46 such that pairs of notches 46 are found on either side of a slot within which the proximal end of arm 32 can be received. A pair of pins 47 project to either side of the proximal end of arm 32. Pins 47 may be received in and engaged with slots 46 to hold arm 32 at a desired position in direction 44. Clip track 45 is optionally mounted in a manner that permits it to swivel from side-to-side. For example, clip track 45 may be mounted to base 34 by a pin that permits clip track 45 to pivot about a vertical axis.

The illustrated embodiment includes an additional hand support member 50 that is disposed on the proximal end of the barrel of syringe 10. Hand support 50 may be compressible. For example, hand support 50 may be made of or have an outer layer of a resiliently-compressible material such as rubber, foam, memory foam, or the like. In other embodiments hand support member 50 is rigid or nearly-rigid. Hand support 50 provides a larger-diameter surface that can be comfortably gripped by a user's hand while the hand retains a relaxed posture. The outer surface of hand support 50 may optionally be textured and/or coated with a resilient material and/or a high friction material so that it can be securely held by a user with reduced grip force. Hand support 50 may have a diameter large enough that it can be comfortably gripped by a user for extended periods of time.

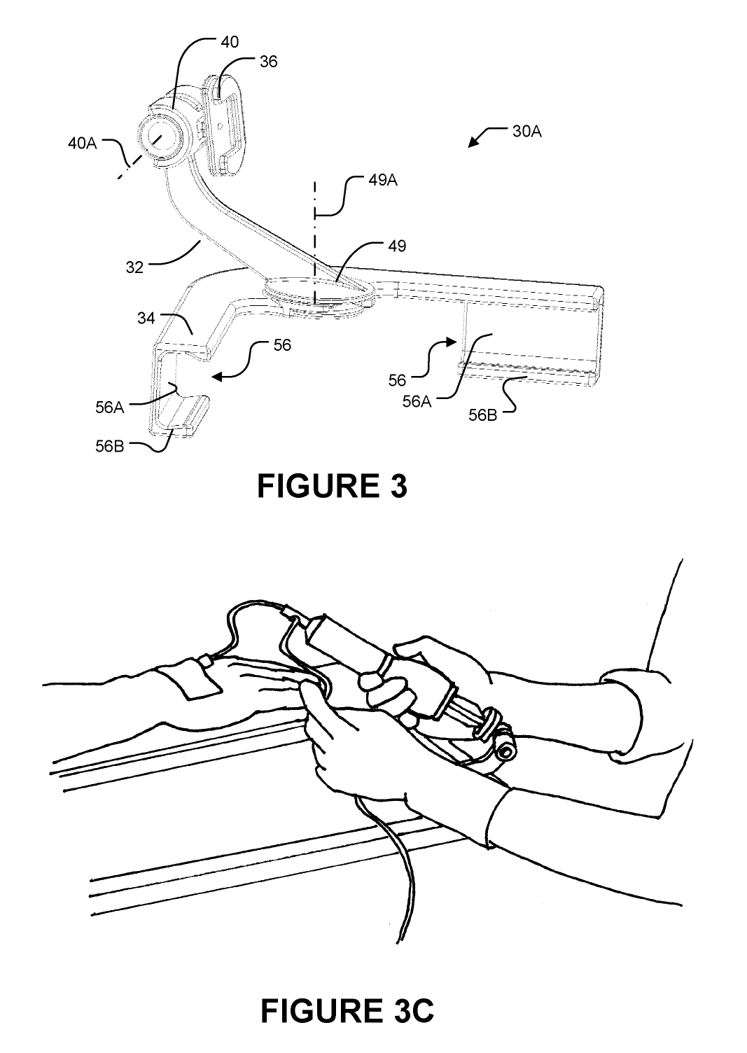

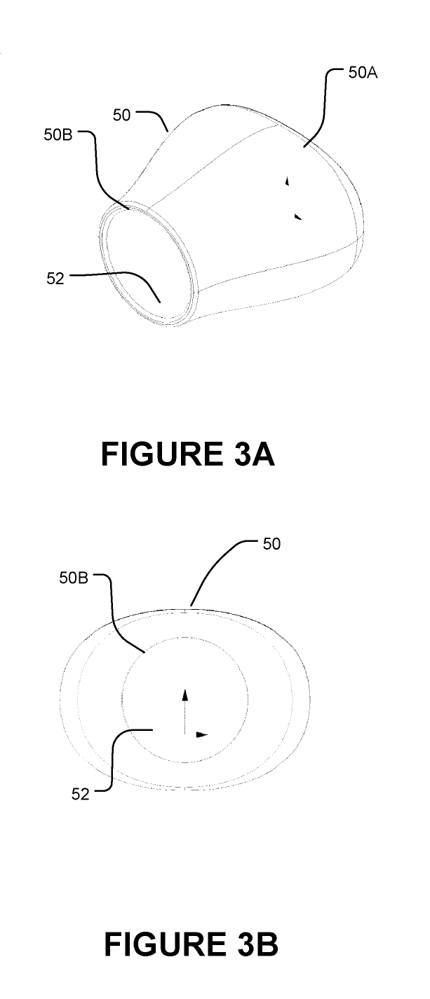

FIG. 3 is a side view of a syringe support 30A that is similar to syringe support 30. In syringe support 30A, arm 32 is coupled to base 34 by a pivotal coupling 49 that permits rotation of arm 32 about an axis 49A. In the illustrated embodiment, axis 49A is at right angles to axis 40A of rotation of pivotal coupling 40.

FIG. 3A shows an example construction for hand support member 50. Hand support 50 includes a central passage 52 that passes over barrel 12 of syringe 10. Passage 52 may be dimensioned such that hand support member 50 lightly grips barrel 12. In the illustrated embodiment, hand support 50 is pear-shaped having a bulging proximal end 50A that tapers down to a thin edge at its distal end 50B. Hand support 50 may be short enough to leave graduations on barrel 12 exposed so that a person using syringe 10 can monitor the progress of the injection.

Hand support 50 may optionally have a slit extending into bore 52. A user can pull the slit open to permit hand support 50 to be placed on a syringe barrel without inserting the distal tip of the syringe through bore 52. The hand support 50 may be made of a resiliently flexible material which allows edges of the slit to be pulled apart sufficiently to pass over the barrel of a syringe from the side and then closes around the barrel of the syringe. This construction may allow hand support 50 to be more easily mounted to the barrel of a syringe and also permits hand support 50 to be mounted in a way that prevents accidental contact with the distal tip of the syringe.

Hand support 50 may be made to have a geometry that is not circularly symmetrical with regard to the longitudinal centerline of passage 52. For example, hand support 50 may be flattened as shown in the end view of FIG. 3B. In some embodiments a ratio of major and minor diameters of hand support 50 at the large end of hand support 50 is about 4:3. for example, the major diameter may be about 2 inches (5 cm) while the minor diameter is about 1½ inches (3¾ cm). In some embodiments both the major diameter and the minor diameter are at least 1¼ inches (about 3.2 cm).

In the embodiment illustrated in FIG. 3, base 34 is configured to be detachably affixable to a tray 38. In the illustrated embodiment, base 34 may be slid into engagement with tray 38. This is achieved by providing a channel 56 which receives an edge 38A of tray 38. In the illustrated embodiment, channel 56 has portions that grip tray 38 on either side of a corner of tray 38. Channel 56 comprises a wall 56A which extends downward from base 34 substantially perpendicular to base 34 and a flange 56B extending continuously or intermittently along the lower edge of wall 56A.

FIG. 3C illustrates a support like that shown in FIG. 3 being used to inject fluids into a patient.

Many alternative structures may be provided which also come within the scope of the invention. For example, arm 32 may be directly mounted to a tray 38 or the like without a separate base 34. Base 34 may attached to tray 38 or another surface in many alternative ways. For example, base 34 may be equipped with one or more of: clamps to grab tray 38; suction cups to adhere to a surface of tray 38; an adhesive for adhesive affixation to tray 38; a magnet to hold base 34 to a metal on or in tray 38; holes accommodating screws or rivets affixing base 34 to tray 38; or the like. Tray 38 may be made to include features for connecting arm 32 directly to tray 38. A syringe support may be configured for mounting to a table, arm-support tray, arm of a chair, bed rail, or the like.

Various alternative structures may be provided to permit horizontal displacement of syringe 10 along a tray 38 or other surface. For example, an arm 32 may be coupled to a traveler member that can be slid along a track comprising a T-shaped member, a T-slot, a bar, or the like that is affixed to tray 38. A clamp, detent mechanism or the like may be provided to lock the traveler at a desired location along the track.

Details of the configuration of pivotal couplings 40 and 49 may also be varied. For example, one or both of couplings 40 and 49 could be provided by a ball joint. A pivotal coupling may optionally be provided with one or more detents, a locking/clamping mechanism or the like to assist in holding the pivotal coupling at a desired angle.

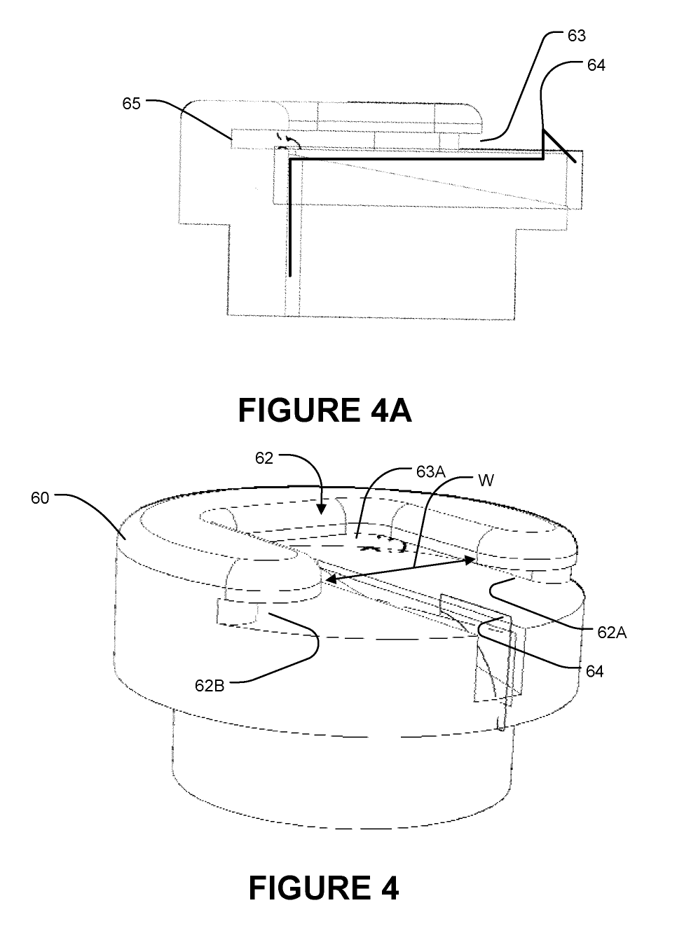

FIG. 4 illustrates a possible implementation of coupling 36. In the illustrated embodiment, coupling 36 comprises a body 60 in which is formed a T-slot 62. Undercut areas 62A and 62B of T-slot 62 are dimensioned to receive corresponding edges of a pad 19 at the proximal end of plunger 14 of a syringe 10 (see FIG. 1). T-slot 62 has an open end 63 into which pad 19 can be slid. The width W of the opening 63A of T-slot 62 is smaller than that of pad 19. A syringe may be coupled to coupling 36 by sliding pad 19 into T-slot 62.

A catch 64 releaseably locks pad 19 in place in T-slot 62. Catch 64 may, for example, be resiliently mounted so that it snaps into place after pad 19 has been fully slid into T-slot 62 but can be manipulated to permit pad 19 to be removed. Catch 64 may, for example, comprise a spring, spring-loaded plunger or the like. In some embodiments, distance between catch 64 and a wall 65 at an opposing end of slot 62 is substantially equal to a width of the interior of slot 62 between walls 62A and 62B. Catch 64 is not required for all applications. For example, for some applications T-slot 62 may be oriented such that open end 63 faces upward and/or the dimensions of T-slot 62 are such that pad 19 is held securely enough for the application without a catch 64.

The dimensions of coupling 36 may be varied to accommodate syringes of different sizes. In some embodiments, coupling 36 is user-detachable from syringe support 30 or 30A so that a user can replace coupling 36 with a coupling suitable for a syringe to be used. For example, connectors dimensioned to attach to the proximal end of the plungers of syringes having capacities of 20 ml, 30 ml and 60 ml may be provided.

A user can operate syringe 10 one-handed after syringe 10 has been coupled to support 30. The user can grasp hand support 50 and pull barrel 12 in the direction of arrow 66 (see FIG. 2) to inject fluid into a patient. The fluid is typically carried into the patient by a short (e.g. 15 cm or 6 inch) section of tubing connected from a fitting of syringe 10 to a needle (not shown) that has been placed in a suitable blood vessel of the patient.

In administering chemotherapy drugs it is sometimes desirable to withdraw fluid back toward syringe 10 as a check to ensure that the needle is still engaged in the blood vessel of the patient. If the needle is properly engaged in the blood vessel of the patient, then withdrawing fluid toward syringe 10 will bring some blood into the tubing which can be observed by the person administering the chemotherapy drugs. One way to withdraw blood into the tubing is to push the barrel of syringe 10 in direction 67 (in cases where the syringe is injecting fluids into a saline line that is connected to deliver the fluids mixed with saline solution to the patient, another way to withdraw blood into the tubing is to interrupt flow in the saline line—e.g. by pinching the saline line with a free hand). Support 30 advantageously supports much or all of the weight of syringe 10 and any drugs in syringe 10. In addition, a user who is operating syringe 10 can grip hand grip 50 of syringe 10 with their entire hand and can move body 12 of syringe 10 in directions 66 and/or 67 using large muscle groups in their arms and shoulders. This contrasts with prior art injection techniques in which muscle groups operating the thumb and fingers are directly used both to support and to operate syringe 10.

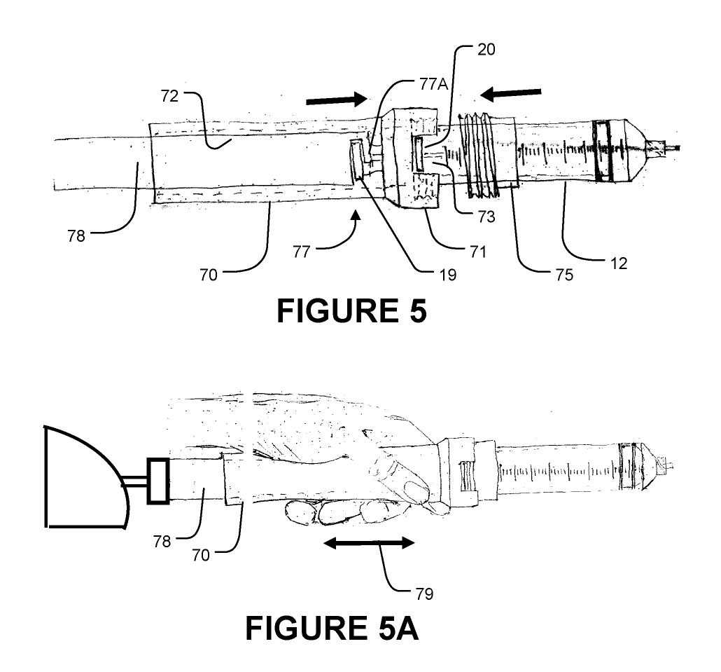

In some embodiments, an extension arm is removably connected to body 12 of syringe 10. An operator may grasp the extension arm instead of body 12 in order to operate syringe 10. In some such embodiments the extension arm slides over a pin that couples to a proximal end of the plunger of the syringe. FIG. 5 shows an example extension arm. In the embodiment of FIG. 5, extension arm 70 comprises a tubular member having a bore 72 extending longitudinally through it. A distal end 71 of tubular member 70 is expanded to receive the proximal end of barrel 12 of syringe 10. Slots 73 are provided to receive flanges 20 of syringe 10. A retaining member 75 holds extension arm 70 to body 12. In the illustrated embodiment, distal end 71 of extension arm 70 has female threads while retaining member 75 has male threads. Retainer member 75 has a bore which slips over the body 12 so that retaining member 75 can be releasably affixed to extension arm 70 thereby holding body 12 in place on the end of extension arm 70.

In the illustrated embodiment, extension arm 70 is designed to be used with a pin 78 that is in turn supported by a suitable support (not shown in FIG. 5). A support of the general type shown in FIG. 2 or 3 could, for example, be used to support pin 78 with modifications to hold pin 78.

Pin 78 is dimensioned to permit it to pass through bore 72 of extension arm 70. A coupler 77 is provided on the distal end of pin 78 for coupling to a proximal end of a syringe plunger. In the illustrated embodiment, coupler 77 comprises a pair of fingers 77A which can engage pad 19 of a plunger 14. Pad 19 is held in place by coupler 77 as long as it is within bore 72 of extension arm 77.

The outer surface of extension arm 70 may be textured and/or coated with a resilient material and/or a high friction material so that it can be readily gripped by a user. Extension arm 70 may have a diameter large enough that it can be comfortably gripped by a user for extended periods of time. A hand support like hand support 50 may optionally be provided on or integrated into extension arm 70.

To attach a syringe 10 to a coupler and extension arm according to this embodiment, pad 19 of the syringe can be slid radially under projecting fingers 77A and extension arm 70 can then be slid along pin 78 until pad 19 and fingers 77A are received within the bore 72 of extension arm 70, thereby preventing removal of pad 19. Retaining member 75 can then be slipped over the end of barrel 12 of syringe 10 and engaged with the threads on distal end 71 of extension arm 70. A user can then operate syringe 10 by sliding extension arm 70 back and forth as indicated by arrow 79 in FIG. 5A. This embodiment can leave the entire length barrel 12 of syringe 10 substantially unobstructed and visible throughout a procedure.

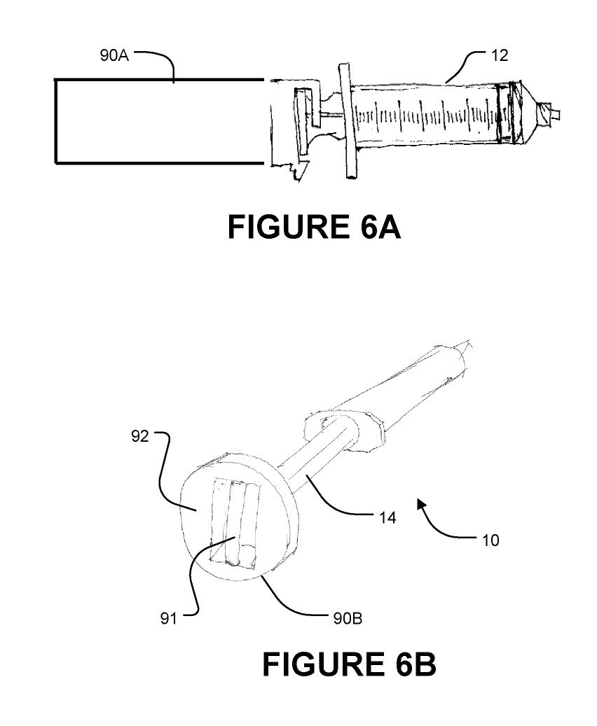

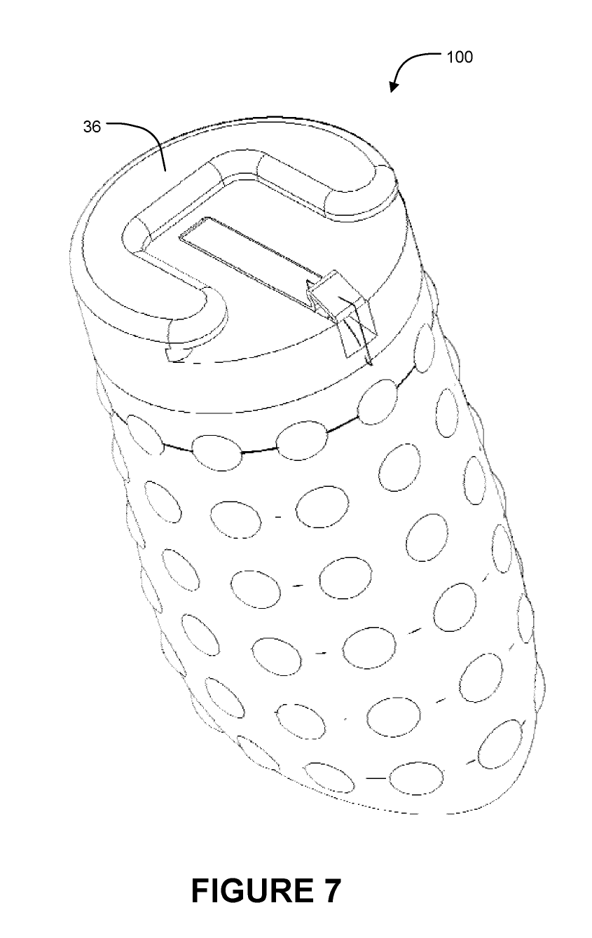

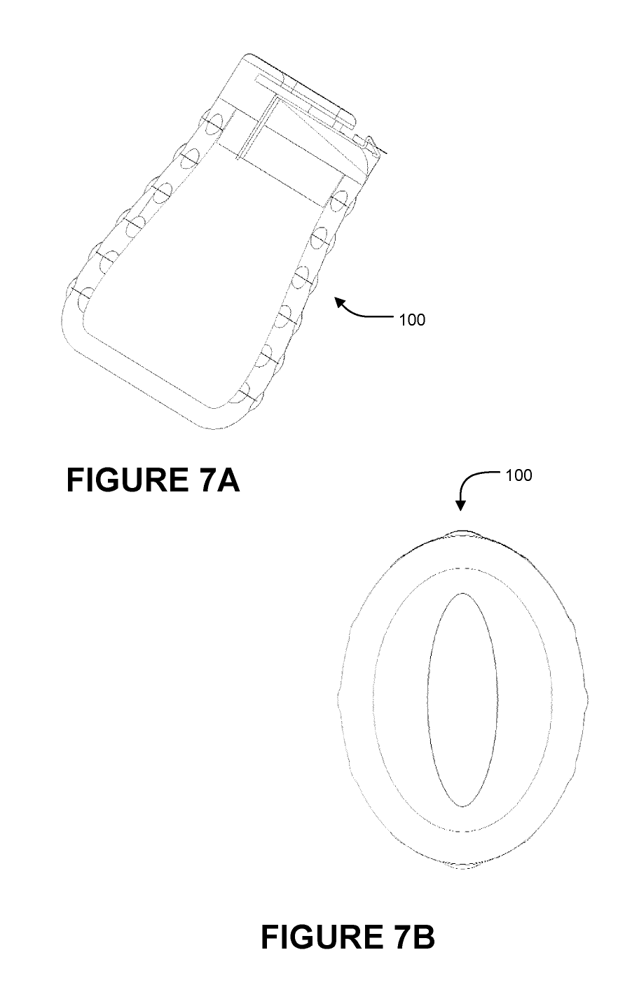

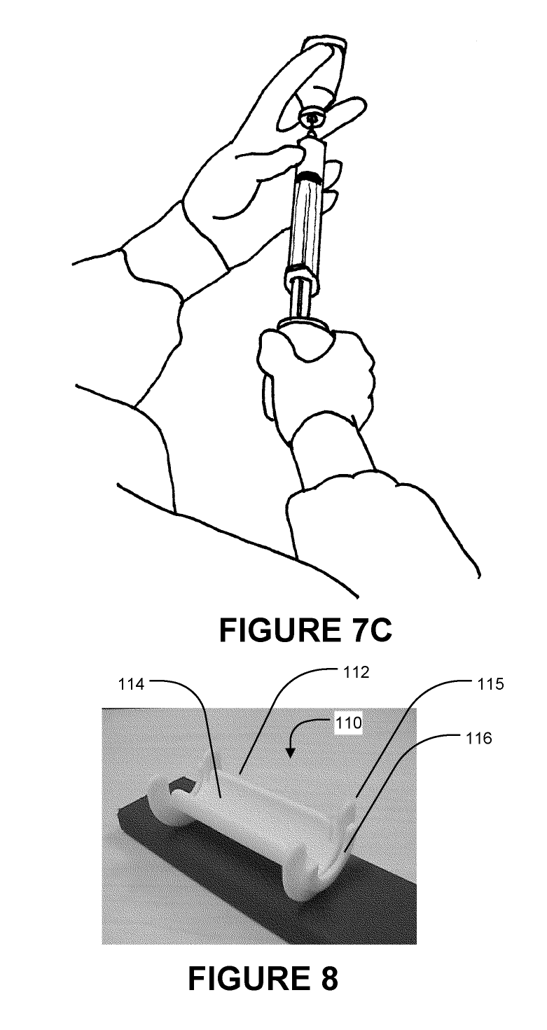

Use of a coupling 36 as illustrated in FIGS. 4 and 4A is not limited to attaching the plunger of a syringe to a support arm. Such couplings (and variations on such couplings) may also be used to attach a variety of useful handles onto standard syringes. Some such handles can facilitate the safe repeated use of syringes. Examples of handles that may be attached to syringes using a coupling like coupling 36 are illustrated in FIGS. 6A, 6B and 7. FIG. 6A shows a rod-like handle adapter 90A. The handle may be cylindrical or have other configurations. For example, the handle may be oval or otherwise flattened in cross-section. FIG. 6B shows an enlarged flat handle adapter 90B which is circular in the illustrated embodiment but could have other shapes as well. Handle adapter 90B provides a generally flat surface 92 oriented generally perpendicularly to the axis of a syringe plunger 14 on which it is mounted. A user can operate a syringe equipped with handle adapter 90B by, for example, pressing handle adapter 90B against their body. A user may apply a pulling force by grasping a recessed handle 91 provided in handle adapter 90B.

FIG. 7 shows a bulb-like hand adapter 100 that may be affixed removably to a proximal end of a syringe plunger. Hand adapter 100 may, for example, be used in a pharmacy setting by a person who needs to repeatedly fill syringes with drugs or other fluids and/or transfer fluids using syringes. A hand adapter 100 has dimensions which allows it to be comfortably gripped in the palm of a user's hand, thereby avoiding the need for the user to exert all of the force needed to drive a syringe plunger with his or her thumb. Hand adapter 100 permits a user to maintain his or her hand and wrist in a neutral posture and also to distribute forces evenly over the entire hand of the user during the operations of filling a syringe and/or dispensing fluid from the syringe.

In some embodiments a proximal end of hand adapter 100 is flat or has feet such that hand adapter 100 can be placed to stand upright on a flat surface with an attached syringe oriented generally vertically. This is useful, for example in the case where a technician fills syringes with medications and a pharmacist checks the doses in the syringes. A technician may fill one or more syringes and then place each syringe in a vertical position standing on the proximal end of hand adapter 100. For example, the syringes may stand on a counter in a fume hood. With the syringes standing upright it is easy for the pharmacist to check the accuracy of the dosage in each syringe.

As shown in FIG. 7B, hand adapter 100 may be non-circular in cross section in a portion near its proximal end (the end away from coupler 36). In the illustrated embodiment, hand adapter 100 is circular or nearly so in its portion near coupler 36 and the cross sectional profile of hand adapter 100 becomes flattened as one moves away from coupler 36. In some embodiments a ratio of major and minor diameters of hand adapter 100 at its widest point is about 4:3. For example, the major diameter may be about 2 inches (5 cm) while the minor diameter is about 1½ inches (3¾ cm). In some embodiments both the major diameter and the minor diameter are at least 1¼ inches (about 3.2 cm). The outer surface of hand adapter 100 may be textured and/or coated with a resilient material and/or a high friction material so that it can be securely held by a user with reduced grip force. Hand adapter 100 may have a diameter large enough that it can be comfortably gripped by a user for extended periods of time.

FIG. 7C shows a hand adapter attached to the plunger of a syringe being filled with fluid from a vial. It can be seen that the hand adapter allows a user to hold the syringe plunger securely without requiring a tight or awkward grip.

Hand adapter 100 may be used on its own or in combination with a hand support member 50 or another ergonomic attachment to the barrel of a syringe that facilitates holding the barrel of the syringe by a user.

An example ergonomic adapter 110 which can be mounted to provide a more comfortable grip on the barrel of a syringe is illustrated in FIG. 8. Adapter 110 comprises a body 112 formed to define a channel 114 dimensioned to receive the barrel 12 of a syringe. Flanges 115 project at either end of body 112. Flanges 115 permit a user to pull or push on the syringe barrel without having to grip the syringe with significant force. Body 112 increases the effective diameter of the barrel of the syringe to permit a user to comfortably grasp the syringe barrel. Flanges 115 provide surfaces against which a user can apply pressure. The presence of these surfaces avoids the need for the user to grip barrel 12 tightly in order to move it in either direction.

Partially circumferential slots 116 at at least one end of adapter 110 are provided to receive projections 20 of a syringe 10. Providing slots 116 at both ends of adapter 110 permits the adapter to be used in either orientation. Channel 114 has a concave configuration which is dimensioned to allow barrel 12 of syringe 10 to be snapped into place within channel 112. Adapter 110 may be placed on a syringe so that the entire scale on the syringe is visible while adapter 110 is in place.

Features of the illustrated embodiments may be combined to yield further example embodiments. Although embodiments as described herein include various features and are intended to provide examples of different ways to practice the invention not all such features are essential. Other example embodiments may be provided by omitting features from the described embodiments.

It can be seen from the above that the invention has various aspects including:
Syringe supports useful, for example, in administering chemotherapy;
Couplings for connecting handles of a wide variety of types to syringe plungers;
Ergonomic hand supports that can be connected to and/or mounted on the barrel of a syringe to provide improved hand position while facilitating good control over the syringe;
Ergonomic handles that may be coupled to a plunger of a syringe to provide improved hand position while facilitating good control over the syringe; and
Chemotherapy treatment chairs that incorporate syringe supports.
   These aspects may be applied individually or in any suitable combinations.

It can be appreciated that at least some embodiments of the invention provide various advantages. For example, some embodiments facilitate delivery of the contents of a syringe with one hand while viewing the graduated measurements on the barrel of the syringe. Some embodiments facilitate delivery of the contents of a syringe with one hand while permitting fluids to be periodically partially drawn back into the syringe barrel. These advantages are useful, for example in the delivery of chemotherapy.

At least some embodiments of the invention provide for the ergonomic delivery of chemotherapy drugs or other fluids using syringes. Ergonomic syringe adapters and/or supports may be applied for dispensing any fluids from standard syringes. Such syringe adapters may help to avoid or alleviate operator pain, discomfort, long term injury and/or disability. Use of such adapters may reduce the risk of repetitive strain injury or other injuries to the health care provider. While the examples given above are in the field of health care, syringe systems as described herein may have application in other fields such as dispensing adhesives, lubricants or other industrial fluids from syringes.

In some embodiments some or all components described herein are intended to be disposable. Such components may be sterilized and provided in sealed pouches. The components may, for example, be made of plastic. In alternative embodiments some or all components of the embodiments described herein are made of metal or other reusable materials.

Some embodiments provide syringe supports that are adjustable to permit support of a syringe in a wide range of positions and orientations. Other simpler embodiments may support a syringe at a fixed location. In example embodiments one or more of the following are adjustable:
the vertical orientation of the syringe relative to a base, other mounting surface or the horizontal (e.g. the syringe may be held horizontally, inclined upwardly or inclined downwardly);
the direction that the syringe is pointing (e.g. the syringe may be pivoted about a vertical or generally vertical axis);
the position at which the syringe is supported (e.g. the syringe may be moved back and forth by adjustment along a horizontal track and/or swung back and forth to a desired position about a vertical axis and/or a mounting point for a syringe support may be adjustable in two dimensions); and
the height of the syringe above a support surface.
   Not all embodiments provide all of these adjustments. As noted above, in some embodiments the position of the syringe is non-adjustable.

Other embodiments of the invention permit a wide variety of handles to be attached to standard syringes. In some embodiments a plurality of handles are provided as a kit. The kit may comprise couplers dimensioned to couple to the proximal ends of the plungers of various sizes of syringe. The handles may, for example, include cylindrical handles, D-handles, large-area flat handles, and the like. The handles may comprise handles of the type illustrated in FIG. 7 for example.

While it is considered advantageous that the ergonomic accessories described herein may be used together with standard syringes of the type that are commonly available and used, it is also possible to make syringes which are built to incorporate such ergonomic accessories.

### INTERPRETATION OF TERMS

Unless the context clearly requires otherwise, throughout the description and the claims:
“comprise,” “comprising,” and the like are to be construed in an inclusive sense, as opposed to an exclusive or exhaustive sense; that is to say, in the sense of “including, but not limited to”.
“connected,” “coupled,” or any variant thereof, means any connection or coupling, either direct or indirect, between two or more elements; the coupling or connection between the elements can be physical, logical, or a combination thereof.
“herein,” “above,” “below,” and words of similar import, when used to describe this specification shall refer to this specification as a whole and not to any particular portions of this specification.
“or,” in reference to a list of two or more items, covers all of the following interpretations of the word: any of the items in the list, all of the items in the list, and any combination of the items in the list.
the singular forms “a”, “an” and “the” also include the meaning of any appropriate plural forms.

Words that indicate directions such as “vertical”, “transverse”, “horizontal”, “upward”, “downward”, “forward”, “backward”, “inward”, “outward”, “vertical”, “transverse”, “left”, “right”, “front”, “back”, “top”, “bottom”, “below”, “above”, “under”, and the like, used in this description and any accompanying claims (where present), depend on the specific orientation of the apparatus described and illustrated. The subject matter described herein may assume various alternative orientations. Accordingly, these directional terms are not strictly defined and should not be interpreted narrowly.

Specific examples of systems, methods and apparatus have been described herein for purposes of illustration. These are only examples. The technology provided herein can be applied to systems other than the example systems described above. Many alterations, modifications, additions, omissions and permutations are possible within the practice of this invention. This invention includes variations on described embodiments that would be apparent to the skilled addressee, including variations obtained by: replacing features, elements and/or acts with equivalent features, elements and/or acts; mixing and matching of features, elements and/or acts from different embodiments; combining features, elements and/or acts from embodiments as described herein with features, elements and/or acts of other technology; and/or omitting combining features, elements and/or acts from described embodiments.

Where a component (e.g. a handle, rod, plunger, coupling, base, chair, etc.) is referred to above, unless otherwise indicated, reference to that component (including a reference to a “means”) should be interpreted as including as equivalents of that component any component which performs the function of the described component (i.e., that is functionally equivalent), including components which are not structurally equivalent to the disclosed structure which performs the function in the illustrated exemplary embodiments of the invention.

It is therefore intended that the following appended claims and claims hereafter introduced are interpreted to include all such modifications, permutations, additions, omissions and sub-combinations as may reasonably be inferred. The scope of the claims should not be limited by the preferred embodiments set forth in the examples, but should be given the broadest interpretation consistent with the description as a whole.

1. Apparatus for supporting a syringe, the apparatus comprising:
a base configured for mounting to a support;
a coupling configured for removable attachment to a proximal end of a plunger of the syringe; and,
an arm connecting the coupling to the base;
whereby a user can operate the syringe with one hand by moving a barrel of the syringe relative to the base.

2. Apparatus according to claim 1 further comprising a patient chair comprising a seat for a patient to sit in and an arm rest mounted adjacent to the seat wherein the base is attached to the arm rest.

3. Apparatus according to claim 1 wherein the arm is pivotally mounted to the base for rotation about a first axis.

4. Apparatus according to claim 3 wherein the first axis is at right angles to the base.

5. Apparatus according to claim 1 wherein the coupler comprises a T-slot arranged to receive a pad at the proximal end of the plunger.

6. Apparatus according to claim 5 comprising a retainer member having a first position wherein the retainer member projects to block removal of the pad from the T-slot and a second position wherein the retainer member does not block removal of the pad from the T-slot.

7. Apparatus according to claim 6 wherein a transverse dimension of the T-slot and a longitudinal dimension between the retainer member and a closed end of the T-slot are substantially equal.

8. Apparatus according to claim 1 wherein the coupler is connected to the arm by a pivotal connection that is pivotal about a second axis extending parallel to the base.

9. Apparatus according to claim 1 wherein the coupler is connected to the arm by a pivotal connection that is pivotal about a horizontal axis.

10. Apparatus according to claim 8 wherein the pivotal connection comprises first and second separable parts, the first part attached to the arm and the second part attached to the coupling.

11. Apparatus according to claim 8 comprising a plurality of couplings dimensioned to couple to a plurality of different sizes of syringe plunger, each of the couplings attached to a corresponding second part of the pivotal connection such that any selected one of the plurality of couplings may be pivotally coupled to the arm at the pivotal connection.

12. Apparatus according to claim 1 comprising a hand support shaped to provide a passage for receiving a barrel of the syringe.

13. Apparatus according to claim 12 wherein the hand support is resiliently compressible.

14. Apparatus according to claim 12 wherein the hand support has a smaller-diameter end and a larger-diameter end and the hand support tapers smoothly toward the smaller-diameter end.

15. Apparatus according to claim 14 wherein the hand support is generally circular in cross-section at the smaller-diameter end and is flattened at the larger-diameter end.

16. Apparatus according to claim 15 wherein the hand support has a major diameter and a minor diameter at the larger-diameter end and a ratio of the major diameter to the minor diameter is approximately 4:3.

17. Apparatus according to claim 16 wherein the major diameter is approximately 2 inches (5 cm).

18. Apparatus according to claim 16 wherein both the major diameter and the minor diameter are at least 1¼ inches (about 3.2 cm).

19. Apparatus according to claim 12 wherein the hand support comprises a compressible foam, memory foam or elastomer material.

20. Apparatus according to claim 12 wherein the hand support has a truncated pear shape.

21. Apparatus according to claim 1 wherein the arm is coupled to the base by a coupling mechanism configured to permit translational movement of the arm relative to the base.

22. Apparatus according to claim 21 wherein the coupling mechanism comprises a track and the arm is mounted to a traveler that is movable along the track.

23. Apparatus according to claim 22 wherein the track is pivotally mounted to the base.

24. Apparatus according to claim 1 wherein the base comprises a channel configured to receive an edge of a table or tray.

25. Apparatus according to claim 24 wherein the base comprises a generally planar support surface and first and second wall sections projecting generally at right angles to the support surface and generally at right angles to one another.

26. Apparatus according to claim 1 comprising a tubular adapter configured to be detachably affixed to a proximal end of a barrel of the syringe and a bore dimensioned to receive the arm and coupling.

27. Apparatus according to claim 26 wherein the adapter comprises a female-threaded sleeve portion having opposed slots dimensioned to receive finger flanges of the syringe barrel and a male-threaded ring having a bore dimensioned slide over the barrel of the syringe.

28. A hand support for use with a syringe, the hand support comprising a body shaped to provide a through-passage for receiving a barrel of the syringe wherein the body has a smaller-diameter end and a larger-diameter end and the hand support tapers smoothly toward the smaller-diameter end.

29. A hand support according to claim 28 wherein the body is resiliently compressible.

30. A hand support according to claim 28 wherein the body is generally circular in cross-section at the smaller-diameter end and is flattened at the larger-diameter end.

31. A hand support according to claim 30 wherein the body has a major diameter and a minor diameter at the larger-diameter end and a ratio of the major diameter to the minor diameter is approximately 4:3.

32. A hand support according to claim 31 wherein the major diameter is approximately 2 inches (5 cm).

33. A hand support according to claim 31 wherein both the major diameter and the minor diameter are at least 1¼ inches (about 3.2 cm).

34. A hand support according to claim 28 wherein the body comprises a compressible foam, memory foam or elastomer material.

35. A hand support according to claim 28 wherein the body has a truncated pear shape.

36. A hand support according to claim 28 wherein the body has a textured outer surface.

37. An accessory handle for a syringe, the accessory handle comprising:
a handle connected to a coupling, the coupling configured to engage a pad at a proximal end of a syringe plunger, the coupling comprising a T-slot and a retainer member having a first position wherein the retainer member projects to block removal of the pad from the T-slot and a second position wherein the retainer member does not block removal of the pad from the T-slot.

38. An accessory handle according to claim 37 wherein a transverse dimension of the T-slot and a longitudinal dimension between the retainer member and a closed end of the T-slot are substantially equal.

39. An accessory handle according to claim 37 wherein the handle comprises a body having a smaller-diameter end joined to the coupling and a larger-diameter end and the body tapers smoothly toward the smaller-diameter end.

40. An accessory handle according to claim 39 wherein the larger-diameter end is flattened.

41. An accessory handle according to claim 39 wherein the smaller-diameter end is circular in cross section.

42. An accessory handle according to claim 40 wherein the body has a major diameter and a minor diameter at the larger-diameter end and a ratio of the major diameter to the minor diameter is approximately 4:3.

43. An accessory handle according to claim 42 wherein the major diameter is approximately 2 inches (5 cm).

44. An accessory handle according to claim 42 wherein both the major diameter and the minor diameter are at least 1¼ inches (about 3.2 cm).

45. An accessory handle according to claim 37 wherein the body has a truncated pear shape.

46. An accessory handle according to claim 37 wherein the body has a textured outer surface.

47. An accessory handle according to claim 37 wherein the body comprises a compressible foam, memory foam or elastomer material.

48. An accessory handle according to claim 37 wherein an end of the body away from the coupling is configured to allow the body to stand on-end while the coupling is coupled to a plunger of a syringe.

49. An accessory handle according to claim 48 wherein the end of the body is flat and parallel to the T-slot.
